(19) United States
(12) Patent Application Publication
(10) Pub. No.: US 2024/0228974 A9
(43) Pub. Date: Jul. 11, 2024

(54) VASCULARIZED ISLET AND PREPARATION METHOD THEREOF

(71) Applicant: NANKAI UNIVERSITY, Tianjin (CN)

(72) Inventors: Deling KONG, Tianjin (CN); Hongmeng MA, Tianjin (CN); Zhuangzhuang YANG, Tianjin (CN)

(73) Assignee: NANKAI UNIVERSITY, Tianjin (CN)

(21) Appl. No.: 18/381,660

(22) Filed: Oct. 19, 2023

(30) Foreign Application Priority Data
Oct. 19, 2022 (CN) 202211277768.4

(51) Int. Cl. C12N 5/071

(52) U.S. Cl. CPC C12N 5/0677; C12N 2506/45; C12N 2513/00

(57) ABSTRACT

Vascularized islets and their preparation method is presented. The vascularized islets are comprised of islet spheroids enclosed within a network of blood vessels. The vascular network is composed of endothelial cells surrounded by smooth muscle cells, forming a tubular vessel with branched structures. The presence of this vasculature significantly enhances the survival of the islets, improves the efficiency of pancreatic precursor cells, islet progenitor cells and R cells, and the increases the synthesis of insulin precursor C-peptide. The procedures for constructing vascularized islets are suitable for long-term in vitro culture of islets.

### CROSS-REFERENCE TO RELATED APPLICATIONS

This application is based upon and claims priority to Chinese Patent Application No. 202211277768.4, filed on Oct. 19, 2022, the entire contents of which are incorporated herein by reference.

### TECHNICAL FIELD

The present invention relates to the field of vascularized organ preparation, in particular to a vascularized islet and its preparation method.

### BACKGROUND OF THE INVENTION

Diabetes is a chronic metabolic disease that affects more than 460 million people worldwide. Type 1 Diabetes (T1D) is characterized by the autoimmune destruction of R cells, resulting in the malfunction in insulin production and persistent hyperglycemia. A potential therapeutic approach for T1D involves transplanting donor islets into the liver via the portal vein, following the Edmonton Protocol. However, achieving independence from insulin requires multiple donors, and there have been instances of immediate loss of graft function post-transplantation. This can be primarily attributed to insufficient revascularization, which hinders graft engraftment. Therefore, it is necessary to develop approaches that improve islet survival and facilitate integration between the graft and the host vasculature to ensure successful transplantation.

Islets are highly vascularized, enabling them to effectively regulate blood glucose levels by secreting insulin. Although the islets constitute only small fraction (1-2%) of pancreatic tissue, they receive high share of the pancreatic blood flow (5-10%). Notably, the density of the islet capillary network is five times greater than that found in the surrounding exocrine pancreatic tissue.

The reciprocal crosstalk between endothelial cells and islet endocrine cells plays a crucial role in islet differentiation and morphogenesis during pancreas development. However, the comprehensive understanding of the interplay between blood vessels and its influence on β-cells in vitro differentiation remains elusive, consequently hampering the differentiation efficiency. Therefore, it is important to establish co-development models involving both blood vessels and pancreatic cells to gain insights into the impact of these reciprocal crosstalk signals on β-cell differentiation.

There is developing a hypoxic-inner core during the in vitro culture period with the increasing the diameter of islet. This condition may lead to increased islet fragmentation and have a detrimental impact on their survival, potentially impeding further research and transplantation. Moreover, after transplantation surgery, the absence of blood flow within islet persists for ten to fifteen days, reducing their survival rate and impairing their ability to function properly. Therefore, it is necessary to reconstruct vascular network within the islet in vitro, as this can prove instrumental in improving the successful of islet transplantation.

In order to solve above-mentioned problems, various approaches have been considered to address the issue of islet revascularization:

Way 1: adding pro-angiogenic factors or inhibiting anti-angiogenic factors to regulate the proliferation, migration, and maturation of endothelial cells.

Although this method has improved the survival and engraftment efficiency of islets, it remains challenging to reconstruct a mature and functional islet-vascular network due to the lack of precise control, the dosage and the duration of vascular production factors post-transplantation.

Way 2: generating mature and functional blood vessels through the activation of endothelial cells or other progenitor cells, which may involve the addition of pre-activated endothelial cells or certain types of endothelial progenitor cell populations.

Nonetheless, it is currently difficult to form stable vessels that faithfully replicate the natural architecture of blood vessels. Consequently, the establishment of vascularized islets in vitro holds substantial importance for enabling rapid revascularization and maintaining the vitality of the islets following transplantation.

### BRIEF SUMMARY OF THE INVENTION

The invention is to reconstruct a vascularized islet characterized by a well-functional perfusion of the vascular network, ultimately enhancing the activity of the islet.

A preparation method of a vascularized islet, comprising the following steps:
Step 1: preparing vascular spheroids and islet spheroids respectively; wherein the vascular spheroids are derived from stem cells with the capacity for vascular lineage cell differentiation, and the islet spheroids are obtained either from the pancreas of a mammal or through differentiated from stem cells with the potential to become islet cells;
Step 2: fuse one or more vascular spheroids with one or more islet spheroids prepared in Step 1 and cultured them to prepare vascularized islets.

Further, the preparation of vascular spheroids in step 1 involves constructing cells which have potential of vascular lineage cells differentiation into spheroids, and then further differentiating into the vascular spheroids; alternatively, vascular lineage cells, which have been differentiated from stem cells under 2D conditions, are aggregated to form vascular spheroids.

Further, the preparation of islet spheroids in step 1 involves constructing cells which have potential of islet cells differentiation into spheroids, and then further differentiating into the islet spheroids; alternatively, islet cells, which have been differentiated from stem cells under 2D conditions, are aggregated to form islet spheroids or the islet spheroids are isolated from mammalian pancreatic tissue.

Preferably, in cases where stem cells possess the capability to differentiate into both vascular lineage cells and islet cells simultaneously, the stem cell-derived embryoid bodies (EBs) are divided into two parts, each part is then differentiated into vascular spheroids and islet spheroids, respectively.

Furthermore, in the fusion process described in step 2, the fusion of vascular spheroids and islet spheroids takes place either under suspended culture conditions or by embedding them into hydrogel for incorporation.

Preferably, the fusion process is under suspended culture conditions.

Further, the ratio of the vascular spheroids and islet spheroids in step 2 is between 1:1 and 1:10.

Further, the number of islet spheroids in step 2 is between 1 and 10.

Further, the number of vascular spheroids in step 2 is between 1 and 5.

Preferably, in step 2, the number of islet spheroids is 5; the number of vascular spheroids is 5.

Preferably, in step 2, the incubation time for 3 to 5 days, the diameter of vascularized islets ranges from 100 μm to 500 μm.

This invention further discloses vascularized islets prepared by using the aforementioned method.

The invention offers the following benefits:
1. The islet spheroids within the vascularized islet are enveloped by vasculature networks;
2. The in vitro survival rate of islet spheroids is promoted;
3. The differentiation efficiency of pancreatic precursor cells is increased;
4. The synthesis efficiency of insulin precursor C-peptide is significantly promoted by the vascular networks;
5. The procedures of vascularized islet construction is economical, efficient and consistently reproducible;
6. The vascularized islet prepared by the present invention has stronger cell activity, and can be cultured for a long time compared with a blood vessel-free islet.
7. The vasculature can promote differentiation of islet progenitor cells and β cells.

### BRIEF DESCRIPTION OF THE FIGURES

FIG. 1. is a schematic illustration the differentiation of islet β cells in Examples 1, 2;

FIG. 2 is a schematic illustrates the differentiation of vascular lineage cells in Examples 1,2;

FIG. 3 shows the morphology of EBs, hiPSC-derived pancreatic precursor cell spheroids and hiPSC-derived vascular spheroids in Example 1;

FIG. 4 is PI staining and statistical analysis of vascularized islet spheroids and islet spheroids in Example 1;

FIG. 5 shows immunofluorescence and statistical analysis of vascular promoting the differentiation of islet progenitor cells in Example 1;

FIG. 6. shows immunofluorescence and statistical analysis of vascular promoting the differentiation of pancreatic R cells in Example 1;

FIG. 7 shows immunofluorescence of vascularized islet spheroids and islet spheroids in Example 1;

FIG. 8 is a brightfield photograph of hiPSC-derived pancreatic precursor cells and endothelial cells in Example 2;

FIG. 9 shows immunofluorescence analysis of hiPSC-derived pancreatic precursor cells and endothelial cells in Example 2;

FIG. 10 is a brightfield photograph of vascularized islet spheroids in Example 2;

FIG. 11. shows immunofluorescence and statistical analysis of vascular promoting the differentiation of islet progenitor cells in Example 2;

FIG. 12. shows immunofluorescence and statistical analysis of vascular promoting the differentiation of pancreatic R cells in Example 2;

FIG. 13. shows immunofluorescence of vascularized islet spheroids and islet spheroids in Example 2;

FIG. 14 is a DTZ staining of isolated mouse islets in Example 3;

FIG. 15 shows the survival rate of vascularized/non-vascularized islet spheroids and the formation of necrosis in the center of non-vascularized islet in Example 3;

FIG. 16. shows immunofluorescence of vascularized islet spheroids and islet spheroids in Example 3;

FIG. 17. shows immunofluorescence analysis of the expression of insulin in vascularized/non-vascularized islet spheroids in Example 3;

FIG. 18. is a morphology photograph of mouse islet cells and hiPSC-derived endothelial cells formed spheroids in Example 4;

FIG. 19. shows immunofluorescence of vascularized islet spheroids and islet spheroids in Example 4.

### DETAILED DESCRIPTION OF THE INVENTION

The following examples are provided to further describe the concrete implementation of the invention. These examples are used solely to clarify the technical aspects of the invention and do not limit the scope of protection.

### Example 1

A preparation method for vascularized islet, includes the following steps:
(1) Human induced pluripotent stem cells (hiPSCs) are dissociated into a single-cell suspension and subsequently transferred to ultra-low attachment plates at a ratio of 1:3, and cultured for one day by using a stem cell culture medium PGM1 to allow the formation of embryoid bodies (EBs).
(2) After 2 days, the EBs were divided into two parts, one part of the EBs initiates the differentiation of islet progenitor cells over an 8-day period using a defined composition for the differentiation system (previous 3 stages). The time point at which differentiation begins are marked as Day 8 before differentiation (shown as Day −8).

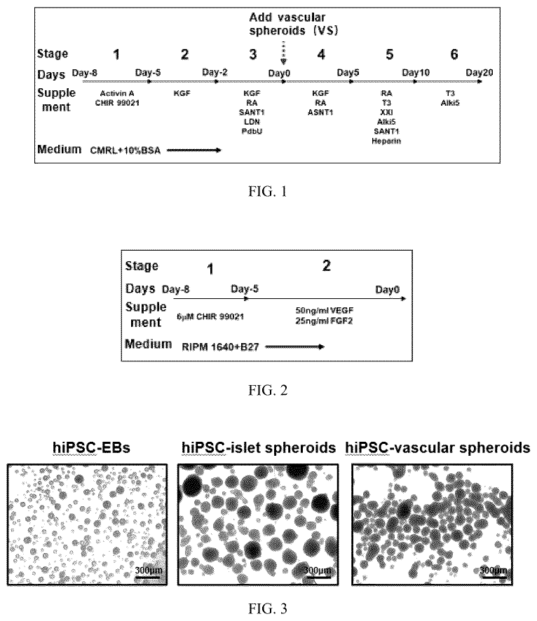

The differentiation stages, the days of differentiation, the culture medium, the differentiation additives and their concentrations for the 8-day period are shown in FIGS. 1-2, and detailed concentration of the additives are shown in Table 1.

TABLE 1
Concentration of the additives

| Additives | Concentration | |
|---|---|---|
| Activin A | 100 | ng/ml |
| CHIR99021 | 3 | mM |
| keratinocyte growth factor (KGF) | 50 | ng/mL |
| Retinoic Acid (RA) | 2 | mM |
| SANT 1 | 0.25 | mM |
| LDN | 200 | nM |
| PdbU | 500 | nM |
| triiodothyronine (T 3) | 1 | mM |
| XXI (Compound E) | 1 | mM |
| Alk5i | 10 | mM |
| Heparin | 10 | μg/mL |

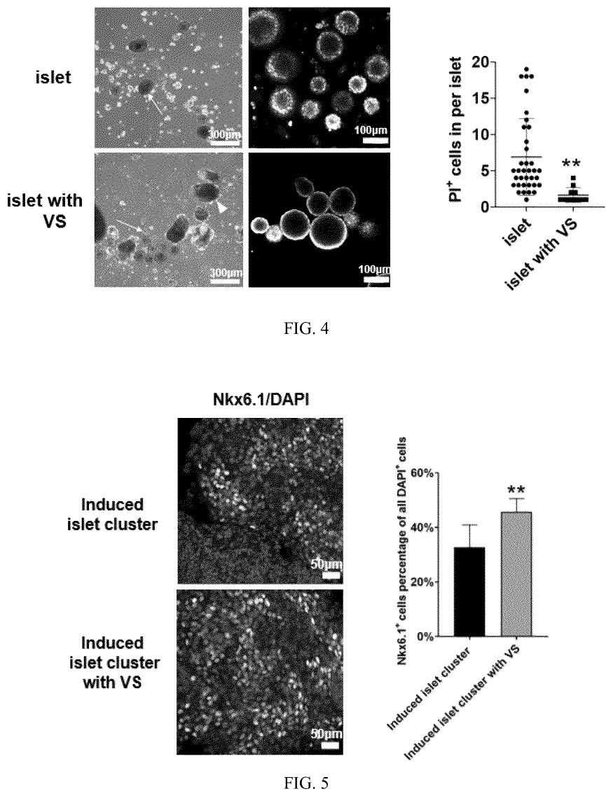

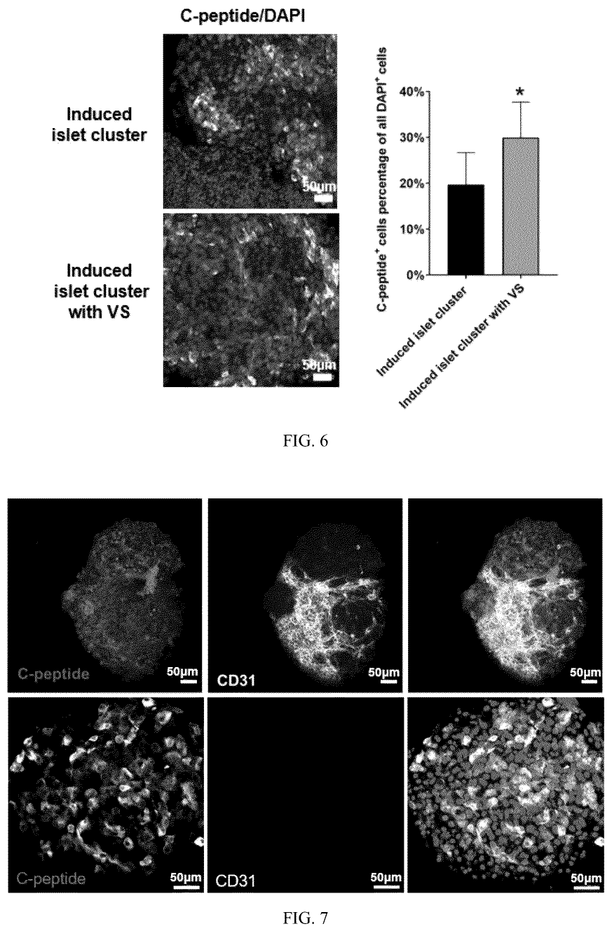

(3) Another part of the EBs was differentiated into vascular spheroids for 8 days. The point of differentiation is denoted as Day 0 (shown as Day 0). The differentiation method of the vascular spheroids is shown in FIG. 2.
(4) The morphology of EBs obtained by Step 1, islet spheroids obtained by Step 2, and vascular spheroids obtained by Step 3 are shown in FIG. 3;
(5) One of vascular spheroid and one of islet progenitor spheroid are implanted into one well of ultra-low attachment 96-well plate (shown in FIG. 4);
(6) After implantation, feed cells with islet spheroids culture medium containing 10% fetal bovine serum (FBS) and place them in the incubator overnight to promote cell survival and facilitate the fusion of cell spheroids.
(7) From day 2 after implantation, the fetal bovine serum (FBS) is removed and the cell viability are assessed using standard PI staining, the fluorescence of PI shows that vascular spheroid co-culture promotes islet spheroid cell survival (as shown in FIG. 4);
(8) Continuing differentiation of islet progenitor cells using the fourth-stage differentiation medium with clear components. After 5 days (on day 5), immunofluorescence staining technique are used to assess the expression of islet progenitor cell specific marker NKX6.1, the results show that the expression of NKX6.1 is significantly increased in the vascularized group (23% vs 16%) (as shown in FIG. 5);
(9) Sustaining the differentiation of islet cells using the seventh-stage differentiation medium with clear components. On day 20, immunofluorescence staining of C-peptide (marker of islet β cells) shows that vasculature promotes the expression of C-peptide in islet spheroids (30% vs 20%) (as shown in FIG. 6).
(10) On the day 20 of forming vascularized islet, immunofluorescence staining of vascular lineage cells (CD31 positive) and islet β cells (C-peptide positive) shows that vascular spheroids are fused with islet spheroids completely and form vasculature networks that cover the islet spheroid (as shown in FIG. 7).
(11) The above results show that the vascular spheroid promotes the survival and viability of islet cells and increases the differentiation efficiency of islet progenitor cells into islet β cells, which proves the vascularization plays an important role in the islet differentiation and development.

In this example, hiPSCs may be substituted for any pluripotent stem cell line or any hESC with similar properties to hiPSC.

### Example 2

A preparation method of a vascularized islet, includes the following steps:
(1) For islet cell differentiation, hESCs (H1 cell line) are disassociated into a single-cell suspensions and then implanted onto Matrigel-coated plates at a seeding density of 1×10⁵/cm², and cultured the hESCs for 1-2 days by using a stem cell culture medium PGM1 until they reach 80-90% confluence. The subsequent islet cell differentiation protocol follows the example 1, with the point of differentiation denoted as Day 8 before differentiation (shown as Day −8).
(2) For endothelial cell differentiation, hESCs (H1 cell line) are disassociated into single-cell suspensions and then implanted onto Matrigel-coated plate at a seed density of 1× 10⁵/cm², and cultured the hESCs for 1-2 days by using a stem cell culture medium PGM1 until they reach 70-80% confluence. The point of differentiation vascular lineage cell is denoted as Day 8 before differentiation (shown as Day −8).

The differentiation stages, the days of differentiation, the culture medium, the differentiation additives and their concentrations for the 8-day period are shown in FIG. 1, and detailed concentration of the additives are shown in Table 1.

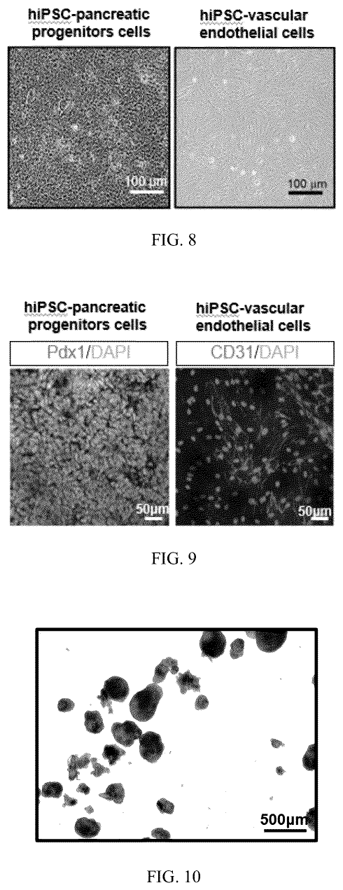

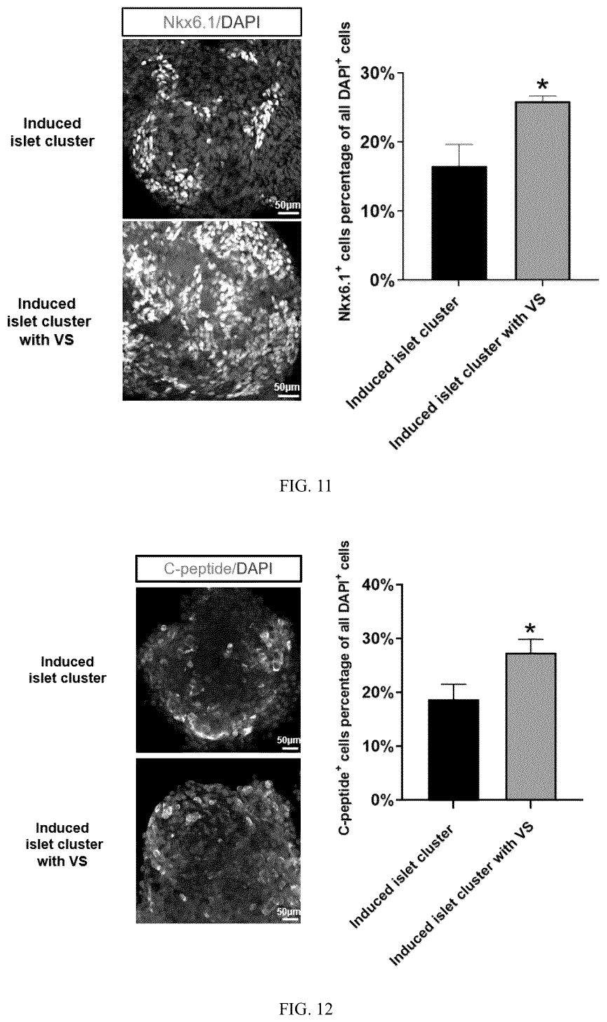

The differentiation method of the vascular spheroids is shown in FIG. 2.
(3) At the stage of differentiation into pancreatic precursor cells (Day 0), analysis the morphology (FIG. 8, left) and the results of immunofluorescence (FIG. 9, left) to demonstrate the extent of differentiation.
(4) The vascular lineage cell is obtained at the day 8 of differentiation through CD31 immunomagnetic beads sorting. The morphology (FIG. 8, right) and immunofluorescence (FIG. 9, right) results are demonstrated highly purified vascular lineage cells.
(5) Islet progenitor cells and endothelial cells are disassociated into cell suspensions and the suspensions are embedding into hydrogel at a ratio of 3:1 for aggregation. The cells are incubated overnight with islet spheroids culture medium containing 10% FBS to promote cell survival and facilitate the fusion of cell spheroids (as shown in FIG. 10).
(6) From day 2 after implantation, the FBS is removed, and place them in a fourth-stage differentiation medium with clearly defined components for further differentiation.
(7) Changing the fourth-stage differentiation medium with clear components to culture the mixture aggregation. On day 5 after implantation, techniques are used to assess the change in islet progenitor cell specific marker NKX6.1 expression, the results show that the expression of NKX6.1 is significantly increased in the vascularized group (25% vs 16%) (as shown in FIG. 11);
(8) Sustaining the differentiation of islet cells using the seventh-stage differentiation medium with clear components. On day 20, immunofluorescence staining of C-peptide shows that vasculature promotes the expression of C-peptide in islet spheroids (27% vs 18%) (as shown in FIG. 12).
(9) On day 20 of forming vascularized islet, immunofluorescence staining of vascular lineage cells (CD31 positive) and islet β cells (C-peptide positive) shows that vascular spheroids are fused with islet spheroids completely and form vasculature networks that cover the islet spheroid (as shown in FIG. 13).
(10) The above results show that the vascular lineage cell promotes the survival and viability of islet cells and increases the differentiation efficiency of islet progenitor cells into islet β cells, which proves the vascularization plays an important role in the islet differentiation and development.

In this example, hiPSCs may be substituted for any pluripotent stem cell line or any hESC with similar properties to hiPSC.

### Example 3

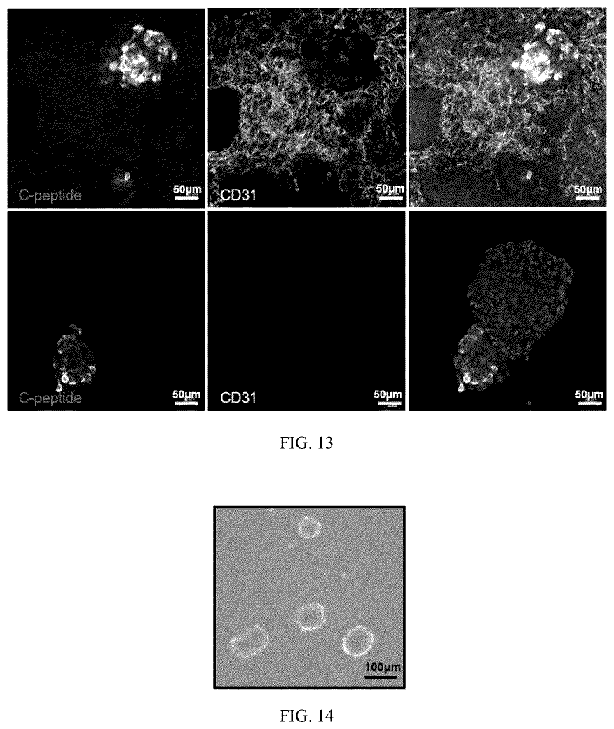

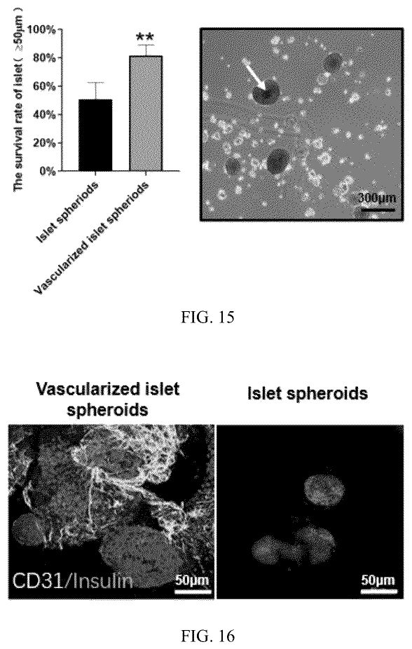

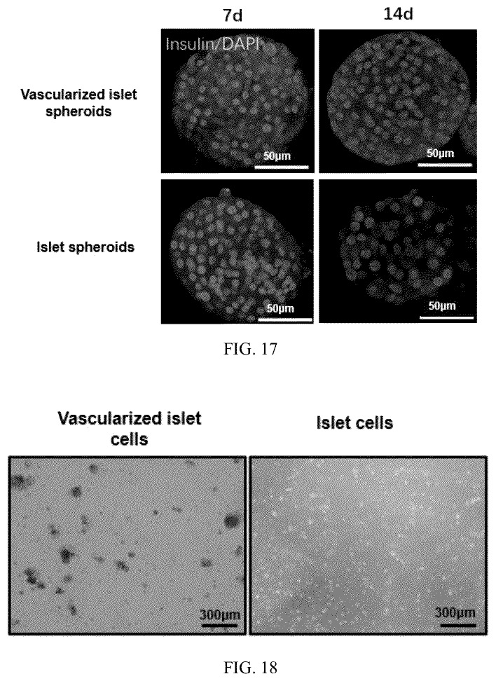

A preparation method of a vascularized islet, includes the following steps:
(1) The vascular spheroids are generated from hiPSCs, prepared according to example 1 or example 2.
(2) The islets are isolated and purified from mouse pancreas. Briefly, following cervical dislocation and sterilization, the abdominal cavity is opened to expose the bile duct. Collagenase-P is then slowly injected through the bile duct to distend the pancreas. The pancreas is then removed and placed in a tube for digestion. After digestion, the tube is shaken and the digestion is terminated. The digested tissue is centrifuged and the pellet is resuspended on a cell strainer. The strained tissue is subjected to gradient density centrifugation to purify the islets. (as shown in FIG. 14).
(3) Pick the vascular spheroids and mouse islets at the ratio of 4:1 and then culture in suspension in vitro.
(4) After 1 week, PI fluorescence staining shows that survival rates are increased in vascularized islets, whereas approximately 40% of the non-vascularized islets are deformed with a larger necrotic center that emerges in the surviving islet spheroids (as shown in FIG. 15).
(5) Immunofluorescence staining result shows that in the presence of vascular spheroids, the expression of islet β cells marker insulin is significantly higher than those in islets without vascular spheroids (as shown in FIG. 16).
(6) These results demonstrate that the addition of the prepared vascular spheroids not only promote the survival of islet spheroids but also maintain the activity of islet β cells (as shown in FIG. 17).

In this example, the isolated and purified islets are not limited to mouse islets but may also be isolated from any mammalian species, including from the human.

### Example 4

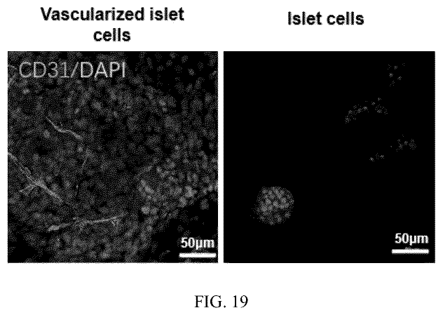

A preparation method of a vascularized islet, includes the following steps:
(1) The endothelial cells spheroids are generated from hiPSC, prepared according to example 1 or example 2.
(2) The islets are obtained by separation and purification from mouse pancreas according to example 3,
(3) The purified mouse islets are digested using 0.25% Trypsin at 37° C. for 3 minutes to obtain single-cell suspension of islet. The cells are then resuspended in a medium containing 10% FBS and count the cell numbers.
(4) Mix the islets cells and endothelial cell spheroids at a ratio of 5:1 under suspended condition;
(5) Incubate the mixed cells for 2 weeks, but only a small percentage of islet cells incorporate with vascular spheroids (as shown in FIG. 18);
(6) Immunofluorescence detection of endothelial cell marker CD31 and islet β cell marker insulin reveals that re-clustered islet cells can coexist with endothelial cells spheroids without losing the expression of their markers. However, the level of vascularization within the islet-vascular spheroids is notably low (as shown in FIG. 19).
(7) According to the above results, we find that only a small portion of the islet cells will be aggregated with endothelial cells spheroids and the formation of a blood vessel network is infrequent. To enhance the formation of vascularized islet, the adjustments can be made to the ratio of islet cells and vascular lineage cells, as well as culture system.

In this example, the isolated and purified islets are not limited to mouse islets but may also be isolated from any mammalian species, including from the human.

It is worth noting that the aforementioned examples are provided solely for the purpose of illustrating the technical scheme of the invention and should not be considered as limiting. While the detailed description of the invention is based on the examples mentioned above, those skilled in the art will understand that the technical scheme outlined in the aforementioned examples may still be subject to modifications and substitutions of some or all of the technical features. Such modifications or substitutions should not depart from the essence of the corresponding technical scheme, as defined within the scope of each example of the invention.

What is claimed is:

1. A preparation method of a vascularized islet, comprising following steps:
step 1: preparing vascular spheroids and islet spheroids respectively; wherein the vascular spheroids are derived from stem cells with a capacity for vascular lineage cell differentiation, and the islet spheroids are obtained either from a pancreas of a mammal or differentiated from stem cells with the potential to become islet cells;
step 2: fusing one or more vascular spheroids with one or more islet spheroids prepared in step 1 and culturing to prepare vascularized islets;
wherein, the preparation of vascular spheroids in step 1 involves constructing cells having a potential of vascular lineage cell differentiation into spheroids, and then further differentiating into the vascular spheroids; alternatively, vascular lineage cells differentiated from stem cells under 2D conditions are aggregated to form vascular spheroids;
the preparation of islet spheroids in step 1 involves constructing cells having a potential of islet cells differentiation into spheroids, and then further differentiating into the islet spheroids; alternatively, islet cells differentiated from stem cells under 2D conditions are aggregated to form islet spheroids or the islet spheroids are isolated from mammalian pancreatic tissue;
in cases where stem cells possess a capability to differentiate into both vascular lineage cells and islet cells simultaneously, human induced pluripotent stem cells (hiPSC)-derived embryoid bodies (EBs) are divided into two parts, each part is then differentiated into vascular spheroids and islet spheroids, respectively.

2. The preparation method according to claim 1, wherein in the fusion process described in step 2, vascular spheroids and islet spheroids are fused under suspended culture conditions or by embedding into hydrogel for incorporation.

3. The preparation method according to claim 1, wherein the ratio of the vascular spheroids and islet spheroids in step 2 is between 1:1 and 10:1.

4. The preparation method according to claim 1, wherein the number of islet spheroids in step 2 is between 1 and 10.

5. The preparation method according to claim 1, wherein, in step 2, the number of islet spheroids is 5, the number of vascular spheroids is 5.

6. The preparation method according to claim 1, wherein, in step 2, the diameter of vascularized islets ranges from 100-500 μm.
